(19) United States
(12) Patent Application Publication
(10) Pub. No.: US 2017/0224815 A1
(43) Pub. Date: Aug. 10, 2017

(54) Method of Preventing and Treating Retinal Microvasculature Inflammation Using C-Met Signaling Pathway Inhibition

(71) Applicant: Nima Tirgan, Morgantown, WV (US)

(72) Inventor: Nima Tirgan, Morgantown, WV (US)

(21) Appl. No.: 15/407,543

(22) Filed: Jan. 17, 2017

Related U.S. Application Data
(63) Continuation of application No. 15/019,000, filed on Feb. 9, 2016, now abandoned.

(51) Int. Cl. A61K 39/395; A61K 31/573; A61K 9/00; C07K 16/22; A61K 38/18

(52) U.S. Cl. CPC A61K 39/3955; C07K 16/22; A61K 38/1833; A61K 9/0053; A61K 9/0048; A61K 9/0019; A61K 31/573; C07K 2317/76; A61K 2039/505

(57) ABSTRACT

The present invention provides methods for preventing retinal microvasculature inflammation in patients with diabetes who are highly susceptible to developing diabetic retinopathy and/or diabetic macular edema. The methods comprise inhibiting C-met signaling pathway by administering a C-met inhibitor alone or in combination with anti-VEGF or steroid medications to diabetic patients. The methods thus provide a surprisingly effective prophylaxis and/or treatment for diabetic retinopathy and/or diabetic macular edema.

### CROSS REFERENCE TO RELATED APPLICATIONS

This application is a continuation of U.S. application Ser. No. 15/019,000, filed Feb. 9, 2016, which is incorporated by reference herein in its entirety.

### FIELD OF THE INVENTION

The present invention relates to methods of preventing and/or treating retinal microvasculature inflammation and, in particular, to the prevention and/or treatment of retinal microvasculature inflammation in individuals susceptible to or afflicted with diabetic retinopathy, diabetic macular edema, diabetic inflammatory macular edema, and clinically significant macular edema by administering compounds that inhibit C-met signaling pathways.

### BACKGROUND OF THE INVENTION

Diabetic retinopathy is a common cause of loss of vision. In fact, it is the leading cause of blindness in those between the ages of 20 to 64 years of age, thus creating a significant burden on healthcare and loss of workforce. Currently, treatments for diabetic retinopathy include medications which inhibit vascular endothelial growth factor (VEGF) and focal/grid photocoagulation in the appropriate clinical circumstances.

Diabetic macular edema, which includes diabetic inflammatory macular edema and clinically significant macular edema, is an accumulation of fluid in the macula, i.e., the part of the retina that controls the most detailed visual abilities, due to leaking blood vessels, and may occur at any stage of diabetic retinopathy. Currently, anti-VEGF medications also are used to treat diabetic macular edema. However, with the anti-VEGF class of medications, there has been a clinical therapeutic ceiling that has been reached, with the need for the development of new therapeutic agents.

Studies have shown that VEGF is a causative factor for diabetic macular edema and neovascularization. The process of neovascularization is termed angiogenesis. There is also evidence that diabetic retinopathy and diabetic macular edema are not limited to angiogenesis but also are a result of a complex inflammatory process. There is an urgent need, therefore, for new therapies to prevent and/or treat diabetic retinopathy and diabetic macular edema.

### SUMMARY OF THE INVENTION

The present invention fulfills this need by providing a method of preventing and/or treating retinal microvasculature inflammation in a subject susceptible to or afflicted with diabetic retinopathy and/or diabetic macular edema and in need thereof. The method comprises of inhibiting the C-met signaling pathway by administering a C-met inhibitor in a therapeutically effective dose to a subject, wherein diabetic retinopathy and/or diabetic macular edema in the subject is prevented and/or treated as a sequelae to the prevention and/or treatment of retinal microvasculature inflammation.

The method comprises of administering the C-met inhibitors via routes which include, without limitation, oral, sublingual, transdermal, subcutaneous, intravenous, intramuscular, intravitreal, transcorneal, eye drops, subconjunctival, sub-tenons, peribulbar, retrobulbar or combinations thereof.

C-Met inhibitors include compounds that inhibit the C-Met receptor, such as monovalent or multivalent antibodies, compounds that block HGF such as C-Met decoy receptor compounds, NK4, a naturally occurring fragment of HGF, which binds to but does not activate the C-Met receptor, thus competitively antagonizing the biological activities of HGF, HGF fragments, C-Met soluble receptor compounds, compounds that inhibit tyrosine kinase activity, compounds that inhibit broad-spectrum kinase activity that have C-Met activity, compounds that inhibit multikinase activity that have C-Met activity, and antisense oligonucleotides.

The C-met inhibitors may be administered once per day, twice per day, three times per day, four times per day, or six times per day for between one month to one year. Alternatively, the administration of the C-met inhibitor may be administered two times per week, once per week, once per month, bi-monthly, between one to two months, every two months, or between once every three months to once every year.

The therapeutically effective dose of the C-met inhibitors to prevent and/or treat retinal microvasculature inflammation ranges between about 0.0001 mg to about 10,000 mg. In an embodiment, the therapeutically effective dose of the C-met inhibitors ranges between about 0.01 mg to about 5.0 mg. The C-met inhibitors may be administered as a single dose, as an extended time release dose, or in a continuous infusion.

The present invention further comprises co-administering the C-met inhibitor with at least one anti-VEGF compound, such as, without limitation, compounds that inhibit VEGF and monoclonal antibodies that inhibit VEGF or VEGF receptor.

The present invention still further comprises co-administering the C-met inhibitor with at least one steroid medication, such as, without limitation, prednisolone acetate, dexamethasone, triamcinolone and methylprednisolone.

The present invention still further comprises co-administering the C-met inhibitor with at least one anti-VEGF compound and at least one steroid medication.

### BRIEF DESCRIPTION OF THE DRAWINGS

FIG. 1 is a flowchart showing the role of the inflammatory process and angiogenesis in diabetic retinopathy and diabetic macular edema.

### DETAILED DESCRIPTION OF THE INVENTION

The present invention provides methods for the prevention and/or treatment of retinal microvasculature inflammation, which, if not prevented and/or treated, can lead to development of diabetic retinopathy and diabetic macular edema in mammals, including humans. The methods disclosed herein provide for the inhibition of the C-Met signaling pathway, using C-Met inhibitor compounds, to prevent and/or treat retinal microvasculature inflammation and thus the development of diabetic retinopathy and diabetic macular edema.

In accordance with the present invention, inhibition of the C-Met pathway includes administration of compounds such as, without limitation, compounds that inhibit the C-Met receptor, such as monovalent or multivalent antibodies, compounds that block HGF such as C-Met decoy receptor compounds, NK4, a naturally occurring fragment of HGF, which binds to but does not activate the c-Met receptor, thus competitively antagonizing the biological activities of HGF, HGF fragments, C-Met soluble receptor compounds, compounds that inhibit tyrosine kinase activity, compounds that inhibit broad-spectrum kinase activity that have C-Met activity, compounds that inhibit multikinase activity that have C-Met activity, and antisense oligonucleotides.

The present invention also includes co-administering a C-met inhibitor with at least one anti-VEGF compound, such as, without limitation, compounds that inhibit VEGF and monoclonal antibodies that inhibit VEGF.

The present invention further includes co-administering a C-met inhibitor with at least one steroid medication, such as, without limitation, prednisolone acetate, dexamethasone, triamcinolone or methylprednisolone.

The present invention still further includes co-administering the C-met inhibitor with at least one anti-VEGF compound and at least one steroid medication.

Those skilled in the art will have knowledge of and be familiar with C-Met pathway inhibitor compounds, anti-VEGF compounds and steroid medications, and thus will have little difficulty selecting the appropriate compounds suitable for use in accordance with the methods of the present invention.

The present invention thus provides the surprising finding that C-Met pathway inhibition is a powerful means to prevent and/or treat inflammation and concomitant injury to microvasculature by the C-Met signaling pathway and leukocytes associated with onset of the spectrum of diabetic retinopathy, which heretofore has not been disclosed or even contemplated.

Diabetic retinopathy (DR) and diabetic macular edema (DME) are common microvascular complications in patients with diabetes and may have a sudden and debilitating impact on visual acuity, eventually leading to blindness. Advanced stages of DR are characterized by the growth of abnormal retinal blood vessels secondary to ischemia. These blood vessels grow in an attempt to supply oxygenated blood to the hypoxic retina. At any time during the progression of DR, patients with diabetes can also develop DME, which is a leading cause of legal blindness in patients with Type II diabetes. Over a 10-year period, non-clinically significant DME and clinically significant DME will develop in 14% and 10% of Americans with known diabetes, respectively.

The early stages of diabetic retinopathy involve the development of retinal microaneurysms, which are dilations of the microvasculature. In addition, capillary non-perfusion and degeneration are further signs of diabetic retinopathy. Eventually, due to non-perfusion, the retina becomes ischemic. The resulting hypoxia results in the release of VEGF. VEGF is thought to be one of the causes of increased leakage from microvasculature and neovascularization. The process of neovascularization is called angiogenesis. There is increased permeability of the vascular endothelium as well as breakdown of the blood-retinal barrier. Breakdown of the blood-retinal barriers results in extracapillary leakage of serum proteins, cytokines and their passage into the retina and vitreous. The leakage of fluid into the macula is termed diabetic macular edema or clinically significant macular edema, which results in the decrease or loss of vision. Eventually, chronic macular edema may result in the loss of photoreceptors that permanently decreases vision despite successful treatment of macular edema.

The spectrum of diabetic retinopathy, which includes but is not limited to diabetic macular edema, diabetic inflammatory macular edema, and clinically significant macular edema, is more complex than the angiogenesis process, as it also involves inflammation. Co-presence of angiogenesis and inflammatory pathways may not be mutually exclusive. Inflammation is a response to injury that includes molecules, cytokines, and the recruitment and/or activation of leukocytes, including polymorphonuclear cells (PMNs). Other leukocytes such as monocytes or macrophages may also be involved. Inflammatory mediators may include nitric oxide, eicosanoids, lipids, adhesion molecules such as ICAM-1, VEGF, cytokines (such as IL-1b and TNFa), complement activation, Fas, NF-kb, CC12, pigment epithelium-derived factor, angiotensin II, and receptors for advanced glycation end products (RAGE).

C-Met (also referred to as MET, MET RTK, hepatocyte growth factor (HGF) receptor, C-met, c-Met, or c-MET) is a member of the semaphorin, plexins, and MET/RON receptor family. C-Met is a cell surface tyrosine kinase receptor. The C-Met receptor has been extensively researched in pre-clinical trials and has recently reached clinical phase studies in the treatment of human cancer.

HGF (also referred to as SF or scatter factor), is the ligand cytokine for the C-Met receptor. It has been shown that HGF in vitro stimulates angiogenesis. In addition, HGF has been shown to be elevated in inflammatory diseases such as fulminant hepatitis, rheumatoid arthritis and diabetic retinopathy, and thus may play a role in inflammation.

Inhibition also may be referred to as blocking, competitively inhibiting, non-competitively inhibiting, diminishing, or reducing. Inhibition of the C-Met pathway refers to the prevention and/or treatment of diabetic retinopathy, which includes diabetic macular edema, diabetic inflammatory macular edema and clinically significant macular edema, using drugs, molecules, compounds, formulations and/or mixtures that prevent, inhibit, block, or reduce the C-Met signaling cascade.

The C-Met pathway also may be referred to as the C-Met signaling pathway, the C-Met/HGF pathway, the C-Met/HGF signaling pathway, C-Met signaling cascade, the C-Met/HGF signaling cascade, the C-Met axis, the C-Met/HGF axis, the C-Met signaling axis, and the C-Met/HGF signaling axis. The C-Met signaling pathway also includes HGF binding to extracellular matrix molecules or to heparin sulfate on the cell surface.

The spectrum of diabetic retinopathy includes diabetic macular edema, which also includes diabetic inflammatory macular edema, clinically significant macular edema, cystoid macular edema, and macular leakage in different clinical and diagnostic contexts.

HGF is synthesized as an inactive precursor (pro-HGF) which then is converted into the active heterodimer by proteolysis. HGF binding occurs at the C-Met receptor. The C-met receptor is located in many tissues, including the retina. Large and microvessel-derived endothelial cells express the C-Met receptor and consequently respond to HGF.

Elevated levels of HGF need to be demonstrated in a diseased state to justify molecular targeting. In human diabetic patients, elevated levels of HGF have been demonstrated in the vitreous, with intravitreal concentrations of HGF shown to be significantly higher in diabetic patients with proliferative diabetic retinopathy (PDR). It also has been demonstrated that levels of aqueous HGF are increased with the stage of diabetic retinopathy. In addition, it has been shown that the concentration of HGF in the aqueous humor of the eye is higher as compared to concentration in serum in all diabetic patients.

Potential targeting of the C-Met/HGF pathway requires preclinical demonstration. Because most laboratory animals lack a macula, it is difficult to create an animal model for diabetic macular edema. However, there are pre-clinical data which show that HGF induces retinal vascular permeability. The effect of HGF on retinal vascular permeability was studied in vivo in male albino Sprague-Dawley rats. HGF was shown to increase retinal vascular permeability in a dose-dependent and time-dependent manner at physiologically relevant concentrations, with HGF signaling possibly independent of VEGF. However, the role of the C-Met signaling pathway, the contribution of inflammation toward the pathophysiology, and the inhibition of the C-Met pathway heretofore has never been shown or suggested.

Inflammation is a response to injury that involves many functional and molecular mediators. Inflammation may have a beneficial effect, but can become pathologic in a diseased state, such as in diabetic retinopathy. PMNs synthesize inflammatory cytokines and growth factors, are produced in the bone marrow, released into the circulatory system, and participate in host defenses by tissue infiltration during an acute inflammatory process. PMNs generate reactive oxygen species (free radicals) as well as release proteolytic enzymes. In fact, PMNs have been shown to store and release HGF. PMNs contain pro-HGF which is processed to active HGF by neutrophil serine protease(s) during the process of degranulation. It also has been shown that PMNs in human blood contain mobilizable HGF, evidence that the C-Met receptor signaling pathway is involved in inflammation.

Specifically with respect to the eye, PMNs have been linked to the pathogenesis of diabetic retinopathy, in its aggregation and adherence to the choriocapillaris of diabetics. In diabetic subjects, PMNs are more rigid, and thus more likely to occlude narrow capillary lumens. PMNs can adhere to endothelial cells and resultant oxygen radical-mediated injury may occur. PMN adherence to the diabetic retinal and/or choroidal circulation can result in an oxidative burst which can damage endothelial cells that result in acellular capillaries. Eventual PMN oxidative burst damages the choriocapillaris resulting in damage to photoreceptors of the retina and visual loss. Heretofore the present invention, there has been no prevention and/or treatment for this pathologic process.

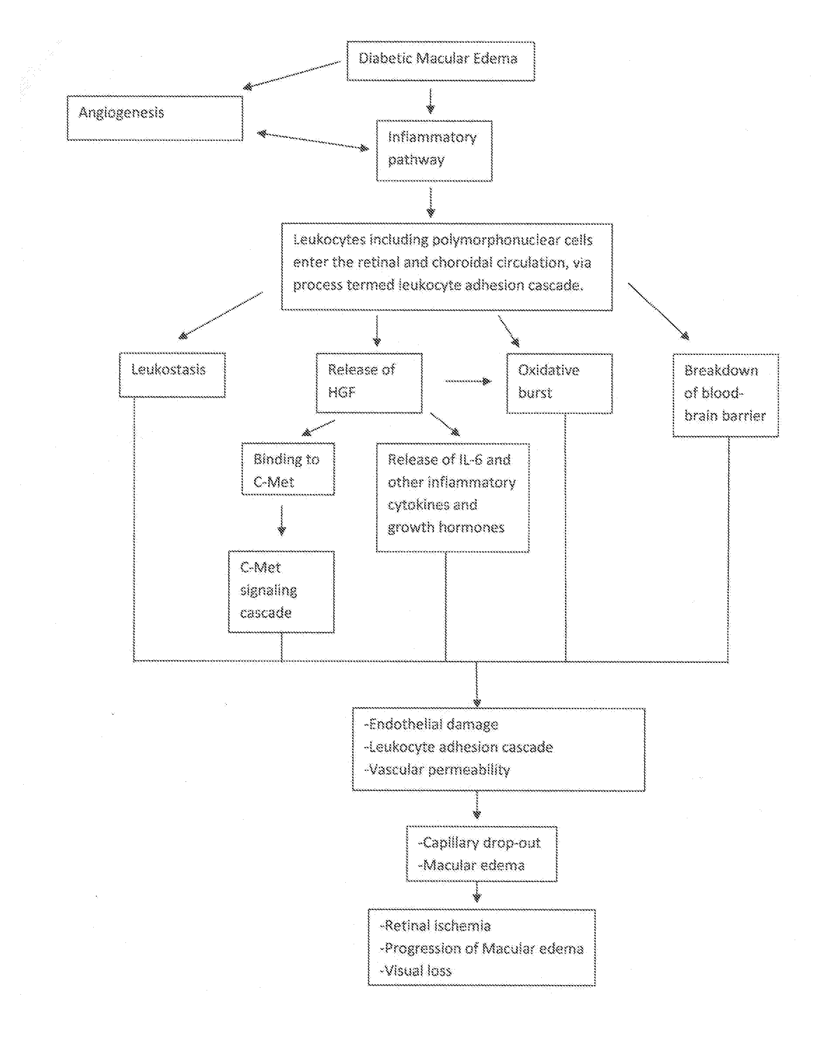

Leukocytes are known to migrate to tissues in a process which may consist of capture/tethering, rolling, activation, adhesion, diapedesis, traversing and migration. This process is described as the leukocyte adhesion cascade. It is not known whether leukocytes complete the whole leukocyte adhesion cascade. Leukocytes may undergo leukostasis in the retinal and/or choroidal circulation. The inflammatory process in diabetic retinopathy and diabetic macular edema, which also includes angiogenesis, is shown in FIG. 1.

PMN's and other leukocytes which aggregate or adhere to the retinal endothelium and/or choroidal vascular endothelium may release HGF (active and/or inactive form) at any point in the leukocyte adhesion cascade or leukostasis. This process is inflammatory and aggravates and/or worsens diabetic retinopathy, diabetic macular edema, diabetic inflammatory macular edema, and clinically significant macular edema via the C-Met signaling pathway. The release of HGF and/or cytokines, and/or growth factors may occur at any point in the leukocyte adhesion cascade or leukostasis. HGF also reaches the vitreous, retinal and choroidal tissues when released from PMNs and other leukocytes. HGF released from leukocytes binds to the C-Met receptors and results in C-Met pathway signaling. HGF may also be released from local eye tissues and then bind to C-Met receptors and result in C-Met pathway signaling. HGF released in affected tissues or extracellular matrix may interact with soluble C-met receptors, membrane bound C-met receptors, or extracellular matrix. Thus, the C-Met pathway signaling is involved in the pathophysiology of diabetic retinopathy, diabetic macular edema and clinically significant macular edema via an inflammatory pathway. The inhibition of the C-Met signaling pathway, using the C-Met inhibitor class of medications, inhibits molecular inflammation, and thus is able to prevent and/or treat of diabetic retinopathy, diabetic macular edema and clinically significant macular edema. In accordance with the present invention, the term “retinal microvasculature inflammation” involves the above described processes.

C-Met inhibitors can be administered in a variety of forms including but not limited to: via a micro-pump, a depot injection, continuous infusion, slow release, local injection such as intravitreal injection, or in combination (either simultaneous or in time). Routes of administration include but are not limited to the following: oral, sublingual, transdermal, subcutaneous, intramuscular, intravitreal, intravenous, transcorneal, eye drops, subconjunctival, sub-tenons, peribulbar, retrobulbar and combinations of thereof.

The compounds provided in the present invention may be formulated as a pharmaceutical formulation, e.g., in a mixture with a pharmaceutical diluent, excipient, and/or carrier. At a minimum, one active compound will be included in the pharmaceutical formulation in association with the appropriate diluent, excipient, and/or carrier. The pharmaceutical diluent, excipient and/or carrier may be selected based on the intended route of administration and standards of pharmaceutical art-accepted practices well known in the art.

The amount of the compound required for successful prevention and/or treatment may vary based on the route of administration, the condition being prevented or treated, body weight, age, patient condition, or a combination of these factors. Alternatively, the amount of compound required for successful prevention and/or treatment may be fixed for a particular condition. The pharmaceutical formulation will contain an effective amount of active compound(s), in combination or alone.

The following dosing intervals may be used in accordance with the invention: multiple daily, daily, every two days up to one month, weekly, monthly, bi-monthly, every 3 months, every six months, yearly, etc.

A subject described herein means any mammal, in particular a human, and may also be referred to as a patient(s), an individual or person(s). The terms “subject” and “subject . . . in need thereof” are interchangeable. The term a “subject . . . in need thereof” can refer to a subject who has already been diagnosed and/or recently treated for a disease state (e.g. diabetic retinopathy or diabetic macular edema). A “subject . . . in need thereof” can refer to a subject who has not yet been treated for the disease state. A “subject . . . in need thereof” can refer to a subject who has been diagnosed with the disease state but who may or may not have initiated treatment due to subclinical or clinical findings. A “subject . . . in need thereof” can also refer to subjects who need preventive treatment, or who have stable, or refractory, or progressive, or an improving disease state. A “subject . . . in need thereof” can also refer to a subject who has a disease state in remission. A “subject . . . in need thereof” may have recently or remotely been treated for the disease state (e.g. 12 months or longer, or within 11, 10, 9, 8, 7, 6, 5, 4, 3, 2, 1 month(s), or about 3, 2, or 1 week(s) or less).

The “effective amount” or “therapeutically effective” terms can be used interchangeably, and refer to an amount, dose, or quantity of pharmaceutical formulation, compound(s), or composition(s) that are adequate or sufficient to result in the desired activity after administration to a subject in need of prevention and/or treatment. The term “therapeutically effective” can refer to an amount, dose, or quantity of pharmaceutical formulation, compound(s), or composition(s) that are adequate or sufficient to prevent, eliminate, cure, delay, reduce, improve or stabilize at least one symptom and/or at least one clinical feature of a condition or disease specified herein, e.g. diabetic retinopathy and diabetic macular edema. The term “therapeutically effective” may also refer to an amount, dose, or quantity of pharmaceutical formulation, compound(s), or composition(s) that are adequate or sufficient to prevent, eliminate, cure, delay, reduce, improve or stabilize at least one radiographic, and/or optical coherence tomography (OCT), and/or fluorescein angiographic, and/or ultrasonic feature of the condition or disease specified herein e.g. diabetic retinopathy and diabetic macular edema. The dose may be administered, e.g., continuously via the methods described, multiple times daily, daily, biweekly, semi-weekly, weekly, less than weekly but more than monthly, monthly, every 2 months, every 3 months etc., to maintain an effective dosage level. The dose may also be administered pro re nata (PRN) or “as needed”, or a variation of treat and extend method. Doses may also be administered in combination as described in the methods herein. The dose(s) may be administered in the following manner but not limited to: continuously by a depot injection, continuous infusion, slow release, oral, sublingual, transdermal, subcutaneous, intramuscular, intravitreal, intravenous, transcorneal, eye drop instillation, sub-tenons, retrobulbar or a combination thereof. In addition, after an initial treatment or treatment period, dosing may be changed to a PRN or “as-needed” to maintain an effective dosage level. Alternatively, dosage and treatment regimen may be changed to a treat and extend protocol. Alternatively, dosage and treatment regimen can remain constant or decreased according to patient response. Alternatively, dosage and treatment regimen can be altered at the discretion of the attending physician.

Doses can range but are not limited to between about 0.0001 mg to about 10,000 mg per dose. In an embodiment, the dose may be in the range of between about 0.01 mg to about 5.0 mg per dose. The dose may be administered in a single dose administration, or by a slow/extended release.

As disclosed herein, “prevention”, “preventing”, “treatment” or “treating” of a condition, disorder, or disease (e.g. diabetic macular edema) includes (1) delaying or preventing the appearance of sub-clinical or clinical symptom(s) in a mammal, who is at risk or predisposed or afflicted with the particular disease state, but who does not experience subjectively the subclinical or clinical symptoms of the particular condition, disorder, or disease, or disorder; and/or (2) inhibition of the condition, disorder, or disease, including reducing, delaying, and/or arresting the development and/or progression of the disease state (or relapse) or at least one clinical, and/or subclinical symptom, and/or imaging finding (e.g. OCT); and/or (3) relieving the disease state (e.g. regression of disease) or at least one sub-clinical, and/or clinical symptom, and/or imaging finding (e.g. OCT); and/or (4) decreasing the severity of disease state in at least one subclinical, and/or clinical symptom, and/or imaging finding (e.g. OCT).

The following disease states can be prevented and/or treated by inhibiting the C-Met pathway in accordance with the invention: exudative macular degeneration (also known as wet age-related macular degeneration), choroidal neovascularization, radiation retinopathy, solar retinopathy, thermal retinopathy, uveitis, anterior uveitis, intermediate uveitis, posterior uveitis, pan-uveitis, white dot spectrum of retinal disorders, macular edema, inflammatory disorders of the eye, retinal arterial occlusive disease, branch retinal artery occlusion, central retinal artery occlusion, branch retinal vein occlusion, hemi-retinal vein occlusion, central retinal vein occlusion, retinal detachment, macular edema secondary to branch or central retinal vein occlusion, retinal tear, proliferative vitreoretinopathy, proliferative diabetic retinopathy, non-proliferative diabetic retinopathy, epiretinal membrane disorders, macular hole, optic neuropathy, retinitis pigmentosa, inherited retinal diseases and/or degenerations, glaucoma, corneal disease, corneal transplant, dry eyes, diabetic macular edema, clinical significant macular edema, and cystoid macular edema.

The methods of the present invention provide a targeted prophylaxis and/or treatment of inflammation associated with diabetic retinopathy and diabetic macular edema. Steroids also target inflammation, but in a non-specific manner. C-met inhibition may be initiated for prevention, as an initial treatment, for maintenance, or if other modes of treatment of macular edema have not resulted in improvement in the disease state. Thus, the present invention provides that in a subset of subjects whose diabetic retinopathy or macular edema does not fully respond to C-met inhibition, combination treatment with steroids or an anti-VEGF medication can be initiated. Combination treatment with steroids represents an improved method of use because steroids act as a non-specific anti-inflammatory agent. Combination treatment with anti-VEGF represents an improved method of use because anti-VEGF inhibits angiogenesis. Combination treatment of diabetic retinopathy, diabetic macular edema, diabetic inflammatory macular edema, and clinically significant macular edema can be started as an initial prophylaxis or treatment or after a period of treatment with C-Met inhibitors. Combination treatment can also be started if the subject is not responding to conventional treatments. Combination treatment also can be started for prevention of disease or for maintenance. Combination treatment can be stopped at any time, for example with a subject's positive clinical response. Combination treatment can be alternated. Alternatively, combination treatment also can represent the use of a single C-Met inhibitor that targets multiple kinases (for example a tyrosine kinase inhibitor that targets C-Met, VEFGR, and KDR). In such a case, adding a second line of treatments can be considered, such as combination with steroid medications.

### EXAMPLES

The present invention is more particularly described in the following non-limiting examples, which are intended to be illustrative only, as numerous modifications and variations therein will be apparent to those skilled in the art.

### Example 1

### Prevention of Inflammation in a Subject Afflicted with Diabetes and Susceptible to Diabetic Retinopathy and Diabetic Macular Edema with Administration of a Tyrosine Kinase Inhibitor via Oral Administration

A subject is diagnosed with diabetes along with non-proliferative diabetic retinopathy. There is no clinical manifestation of diabetic macular edema in the right eye. There may be evidence of no diabetic macular edema, pre-clinical macular edema, or only mild macular thickening of less than 300 microns on optical coherence tomography (OCT) in this right eye. The contralateral left eye has severe diabetic retinopathy not amenable to treatment. A tyrosine kinase inhibitor which targets both the VEGF receptor and the C-Met receptor is given orally in a dose of 25 mg to prevent the development of diabetic macular edema in the right eye. The subject is then followed every 3 months. Re-injection is given per the discretion of the attending physician.

### Example 2

### Treatment of Subject with Diabetic Macular Edema with a Monoclonal Antibody to Hepatocyte Growth Factor via Intravitreal Injection

A subject is diagnosed with clinical manifestations of diabetic macular edema. To inhibit the C-met signaling pathway that is implicated in the inflammatory component of this disease, a monoclonal antibody which binds to HGF and thus prevents binding of HGF to the C-met receptor is administered at a dose of 1.0 mg via intravitreal injection. After three months of monthly injections, the subject's condition improves. Treatment then is continued once per month for at least three more months, after which time the subject's condition is reevaluated. At that time, the subject's diabetic macular edema has resolved and the injections are stopped.

### Example 3

### Treatment of Subject with Diabetic Macular Edema with a Monoclonal Antibody to Hepatocyte Growth Factor via Sub-Tenon's Injection

A subject is diagnosed with clinical manifestations of diabetic macular edema. To inhibit the C-met signaling pathway that is implicated in the inflammatory component of this disease, a monoclonal antibody which binds to HGF and thus prevents binding of HGF to the C-metreceptor, is administered at a dose of 5.0 mg via sub-tenon's injection. After three months of once per month injections, the subject's condition stabilizes and improves. Treatment is discontinued while frequently monitoring the subject's condition. The subject develops recurrence of disease at 6 months. Treatment is then re-started with once per month injections at a higher dose of 10.0 mg and with close follow-up.

### Example 4

### Treatment of Subject with Diabetic Macular Edema with a Monoclonal Antibody to the C-Met Receptor via Intravenous Injection

A subject is diagnosed with clinical manifestations of diabetic macular edema. To inhibit the C-met signaling pathway that is implicated in the inflammatory component of this disease, a monoclonal antibody which binds to the C-Met receptor thus prevents binding of HGF to the C-met receptor is administered at a dose of 50 mg via a single intravenous injection. After three months, the subject's condition does not improve, and a repeat dose of 50 mg is given via intravenous injection. After another 3 months, there is improvement and re-injection is performed to continue improvement. Treatment is then continued as long as there is diabetic macular edema, every 3 months.

### Example 5

### Treatment of Subject with Diabetic Macular Edema with NK4 via Intravitreal Injection in Combination with an Anti-VEGF Compound via Intravitreal Injection

A subject is diagnosed with clinical manifestations of diabetic macular edema. This subject was previously treated with focal laser and ten anti-VEGF injections with marginal improvement in diabetic macular edema over a one year period. Because of the subject's multiple treatments and chronic duration of disease, the subject is treated with combination treatment which includes the C-met inhibitor NK4, a naturally occurring fragment of HGF which binds to but does not activate the c-Met receptor, and thus competitively antagonizes the biological activities of HGF, and an anti-VEGF compound that inhibits VEGF. NK4 is administered at a dose of 4.0 mg, and the anti-VEGF compound is administered at a dose of 2.0 mg, both by intravitreal injection. A 30 gauge needle is used to administer the first medication, and only the syringe is changed to administer the second medication. A total volume of 0.1 cc is given. The subject is given the same combination treatment monthly for 3 months, at which point there is improvement and the anti-VEGF compound is stopped. The subject then is maintained on the C-met inhibitor for 3 monthly injections. After 3 more months, there is resolution of the diabetic macular edema and the subject is given intravitreal NK4 on a PRN basis.

### Example 6

### Treatment of Subject with Diabetic Macular Edema with a C-Met Tyrosine Kinase Receptor Inhibitor via Oral Administration in Combination with Steroid Medication Dexamethasone via Intravitreal Injection

A subject is diagnosed with clinical manifestations of diabetic macular edema. This subject was previously treated with focal laser and administration of a monoclonal antibody that inhibits VEGF with marginal improvement in diabetic macular edema over a 1 year period. Because of the subject's multiple treatments and chronic duration of disease, the subject is treated with a combination treatment of a C-met receptor tyrosine kinase inhibitor and the steroid medication dexamethasone. The C-Met inhibitor is given via oral administration at a dose of 360 mg by mouth twice daily for one week. Dexamethasone is given via intravitreal route as an intravitreal implant at a dose of 0.7 mg. Following the intravitreal injection, the subject is started on the C-met inhibitor the same day for seven days. The subject is given another seven day course of the C-met inhibitor at the 3 month interval. At the 6 month interval, the subject shows resolution of diabetic macular edema. The subject's treatment is stopped. The subject is then monitored every 3 months.

### Example 7

### Treatment of Subject with Diabetic Macular Edema with NK4 via Transcorneal Drops in Combination with a Monoclonal Antibody VEGF Inhibitor via Intravitreal Injection and Triamcinolone Acetonide via Intravitreal Injection

A subject is diagnosed with clinical manifestations of diabetic macular edema. To inhibit the C-met signaling pathway that is implicated in the inflammatory component of this disease, the C-met inhibitor NK4 is administered at a dose of 5.0 mg via transcorneal drops twice daily, a monoclonal antibody VEGF inhibitor at a dose of 1.25 mg/0.05 mL via intravitreal injection, and the steroid triamcinolone acetonide at a dose of 4 mg in 0.1 mL intravitreal dose. Triamcinolone acetonide is not given the same day, but seven days later. After 3 months, there is improvement in the diabetic macular edema. For further improvement, the same regimen is repeated, and the subject is re-evaluated in 3 months. At that time, the subject only has minimal residual diabetic macular edema. At this point, the subject is only continued on maintenance NK4 transcorneal drops for 6 months. At re-evaluation, the subject shows no sign of diabetic macular edema and treatment with trans-corneal drops is discontinued.

While the invention has been particularly shown and described with reference to embodiments described above, it will be understood by those skilled in the art that various alterations in form and detail may be made therein without departing from the spirit and scope of the invention, as defined by the appended claims.

1-14. (canceled)

15. A method of preventing and/or treating retinal microvasculature inflammation in a subject susceptible to or afflicted with diabetic retinopathy and/or diabetic macular edema and in need thereof, comprising inhibiting C-met signaling pathway by administering a C-met inhibitor in a therapeutically effective dose to the subject, wherein diabetic retinopathy and/or diabetic macular edema in the subject is prevented and/or treated as a sequelae to the prevention and/or treatment of retinal microvasculature inflammation.

16. The method of claim 1, wherein the C-met inhibitor comprises compounds that inhibit the C-Met signaling pathway selected from the group consisting of monovalent or multivalent antibodies, compounds that block hepatocyte growth factor (HGF) including C-Met receptor compounds, NK4, a naturally occurring fragment of HGF, which binds to but does not activate the C-Met receptor, thus competitively antagonizing the biological activities of HGF; HGF fragments; C-Met soluble receptor compounds; compounds that inhibit tyrosine kinase activity; compounds that inhibit broad-spectrum kinase activity that have C-Met activity; compounds that inhibit multikinase activity that have C-Met activity; and antisense oligonucleotides.

17. The method of claim 1, wherein the administration of the C-met inhibitor is selected from the group consisting of oral, sublingual, transdermal, subcutaneous, intramuscular, intravitreal, intravenous, transcorneal, subconjunctival, sub-tenons, peribulbar, retrobulbar and combinations thereof.

18. The method of claim 1, wherein the administration of the C-met inhibitor is administered once per day, twice per day, three times per day, four times per day or six times per day

19. The method of claim 18, wherein the administration of the C-met inhibitor is administered to the subject for between one month to one year.

20. The method of claim 1, wherein the administration of the C-met inhibitor is administered two times per week, once per week, two times per month, once per month, bi-monthly, or between once every three months to once every year.

21. The method of claim 1, wherein the therapeutically effective dose ranges between about 0.0001 mg to about 10,000 mg.

22. The method of claim 1, wherein the therapeutically effective dose ranges between about 0.01 mg to about 5.0 mg.

23. The method of claim 1, wherein the therapeutically effective dose is administered as a single dose or as an extended time release dose.

24. The method of claim 1, further comprising co-administering the C-met inhibitor with at least one anti-vascular endothelial growth factor (anti-VEGF) medication.

25. The method of claim 24, wherein the at least one anti-VEGF medication comprises compounds that inhibit VEGF or monoclonal antibodies that inhibit VEGF or VEGF receptor.

26. The method of claim 1, further comprising co-administering the C-met inhibitor with at least one steroid medication.

27. The method of claim 26, wherein the at least one steroid medication comprises prednisolone acetate, dexamethasone, triamcinolone acetonide or methylprednisolone.

28. The method of claim 1, further comprising co-administering the C-met inhibitor with at least one anti-VEGF medication and at least one steroid medication.
